(19) United States
(12) Patent Application Publication
(10) Pub. No.: US 2003/0187591 A1
(43) Pub. Date: Oct. 2, 2003

(54) Method of and apparatus for genomic analysis, and computer product

(75) Inventors: Osamu Tezuka, Nagano (JP); Mitsuo Itakura, Tokushima (JP); Shuuichi Shinohara, Kawasaki (JP)

Correspondence Address: STAAS & HALSEY LLP, SUITE 700, 1201 NEW YORK AVENUE, N.W., WASHINGTON, DC, 20005, US

(73) Assignees: FUJITSU LIMITED, Kawasaki (JP); MITSUO ITAKURA, TOKUSHIMA (JP)

(21) Appl. No.: 10/270,197

(22) Filed: Oct. 15, 2002

(30) Foreign Application Priority Data
Mar. 27, 2002 (JP) 2002-089516

(51) Int. Cl. G06G 7/48; G06G 7/58; G06F 19/00; G01N 33/48; G01N 33/50

(52) U.S. Cl. 702/20; 703/11

(57) ABSTRACT

Genomic sequence information consisting of four base sequences is input. It is determined whether there is a sequence portion in which any one of the bases is arranged continuously for, for example, ten in the input information. If there is such a sequence portion, base sequence information consisting of a predetermined number of bases continuously arranged forwards and rearwards of the sequence portion is extracted, and the extracted base sequence information is output.

### BACKGROUND OF THE INVENTION

1) Field of the Invention

The present invention relates to a technology for searching disease-related candidate genes.

2) Description of the Related Art

Conventionally, as a polymorphic marker for genetic polymorphism analysis for searching disease-related candidate genes using a difference or similarity of individual genetic information, single-nucleotide polymorphism (SNP) or a micro-satellite marker is generally used. More specifically, in the SNP, many samples are extracted through direct sequencing, and the micro-satellite marker is formed by repetition of generally from 2 to 4 base units.

The polymorphic marker can be used for correlation analysis in which the position of genes related to a disease is statistically guessed from the correlation between a classification method using patterns of the polymorphic marker and a classification method using the existence or nonexistence of a disease, or for various genetic statistical analyses such as linkage analysis in which the correlation between a propagation method of patterns of the polymorphic marker and a propagation method of a disease from parents to children is studied using the family information and the position of genes related to the disease is guessed. Preparation of SNPs database is now in progress globally as a polymorphic marker for genetic polymorphism analysis.

In the conventional art described above, however, if it is tried to actually use these database, in many cases, the SNPs data in the objective field has not yet been prepared sufficiently, and search of SNPs must be specially performed. It is practically difficult to newly start the SNPs search, in view of equipment and systems, and there is also a problem in that huge cost and time are required.

On the other hand, the micro-satellite marker which can be extracted relatively easily from the genomic sequence has a problem in that the number of markers is small, and the analytical density decreases as compared to the SNPs. Further, there are many polymorphic patterns, and it is considered that a mutation rate is considerably high as compared to the SNPs. If it is a marker in which many mutations have occurred, there is a problem in that noise (mutation) is large and the power of the test decreases, as the marker for genetic polymorphism analysis for searching disease-related candidate genes from the correlation between inheritance and disease.

### SUMMARY OF THE INVENTION

It is an object of this invention to provide a genomic analysis method, a genomic analysis program, a genomic analysis apparatus, and a genomic analysis terminal unit capable of finding a polymorphic marker for identifying a disease-related candidate gene quickly and efficiently with a nearly the same degree of accuracy as that of the SNPs, without using the SNPs.

The present invention provides the genomic analysis method, the genomic analysis program, and the genomic analysis apparatus. The genomic analysis method comprises inputting genomic sequence information including four base sequence of adenine (A), thymine (T), guanine (G) and cytosine (C), and determining whether there is a sequence portion in which either one of the four bases and the same base is arranged continuously for a plurality of numbers in the input genomic sequence information. The method also comprises, when it is determined there is the sequence portion in which the same base is arranged continuously for a plurality of numbers (for example, 10), obtaining the information relating to the position of the sequence portion in the genomic sequence, extracting at least one of the base sequence information, of the base sequence information comprising a predetermined number of bases continuously arranged forwards of the sequence portion, and the base sequence information comprising the same number of bases as or a different number of bases from the predetermined number, which are continuously arranged rearwards of the sequence portion, and outputting the obtained information relating to the position and the extracted base sequence information.

According to the above aspect, the sequence portion in which the same base is arranged continuously for a plurality of numbers (for example, 10) can be searched relatively easily, and by using the sequence portion as a mark, the base sequence in the vicinity of the sequence portion having high possibility that the disease-related candidate genes are included, can be easily identified at the nearly same degree of accuracy as that of the SNPs.

These and other objects, features and advantages of the present invention are specifically set forth in or will become apparent from the following detailed descriptions of the invention when read in conjunction with the accompanying drawings.

### BRIEF DESCRIPTION OF THE DRAWINGS

FIG. 1 is an explanatory diagram which shows the general outline of analysis of disease-related candidate genes, including the genomic analysis method according to an embodiment of this invention,

FIG. 2 is a block diagram which shows one example of hardware configuration of a computer 102, being a genomic analysis apparatus according to the embodiment of this invention,

FIG. 3 is a block diagram which shows one example of a functional structure of the genomic analysis apparatus according to the embodiment of this invention,

FIG. 4 is an explanatory diagram which shows one example of the contents of genomic sequence information,

FIG. 5 is an explanatory diagram which shows one example of the contents of polymorphic marker information,

FIG. 6 is a flowchart which shows the processing procedure of the genomic analysis apparatus according to the embodiment of this invention,

FIG. 7 is a flowchart which shows the processing procedure of the analysis of disease-related candidate genes, including the genomic analysis method according to the embodiment of this invention,

FIG. 8 is an explanatory diagram which shows one example of application of the polymorphic marker information, and

FIG. 9 is another explanatory diagram which shows one example of application of the polymorphic marker information.

### DETAILED DESCRIPTION

Embodiments of the genomic analysis method, the genomic analysis program, and the genomic analysis apparatus according to this invention will be explained in detail with reference to the accompanying drawings.

General outline of analysis of disease-related candidate genes:

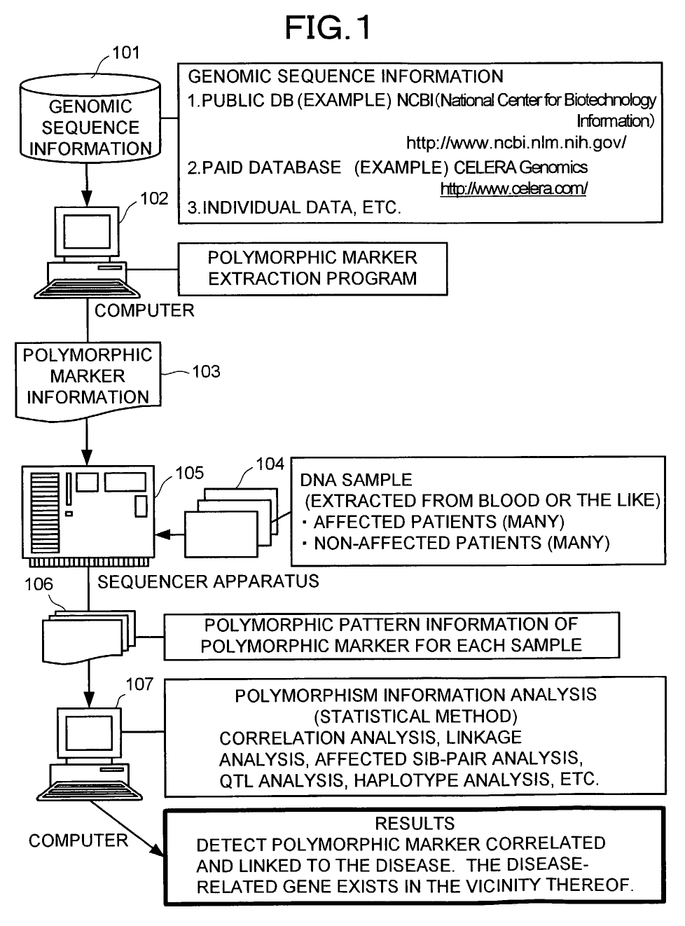

The general outline of analysis of disease-related candidate genes including the genomic analysis method according to the embodiment of this invention will be explained below. FIG. 1 is an explanatory diagram which shows the general outline of analysis of disease-related candidate genes, including the genomic analysis method according to the embodiment of this invention. In this figure, reference numeral 101 is genomic sequence information. The genomic sequence information 101 may be collected, for example, from public database (e.g., NCBI (National Center for Biotechnology Information) or from paid database (for example, CELERA Genomics). Alternatively, individual data may be used for the genomic sequence information 101.

This genomic sequence information 101 is input to a computer 102 in which a polymorphic marker extraction program is installed. This computer 102 is the genomic analysis apparatus according to this embodiment. As the analysis result, the polymorphic marker information 103 is output. This polymorphic marker information 103 and DNA samples 104 extracted from bloods of many affected patients and non-affected patients are input to a sequencer apparatus 105. As the result thereof, polymorphic pattern information 106 of polymorphic markers for each sample is obtained.

The polymorphic pattern information 106 is input to a polymorphism information analysis apparatus (computer) 107 to perform haplotype analysis for studying the correlation between the haplotype polymorphic pattern built from a plurality of SNPs and the presence of a disease, and various other analyses such as correlation analysis, linkage analysis, affected sib-pair analysis, QTL analysis, and haplotype analysis. As the result, a polymorphic marker correlated and linked with the disease is detected. Then, by analyzing the sequence in the vicinity of the detected polymorphic marker, it is seen that there is a disease-related candidate gene in the sequence in the vicinity thereof.

Hardware configuration of the genomic analysis apparatus:

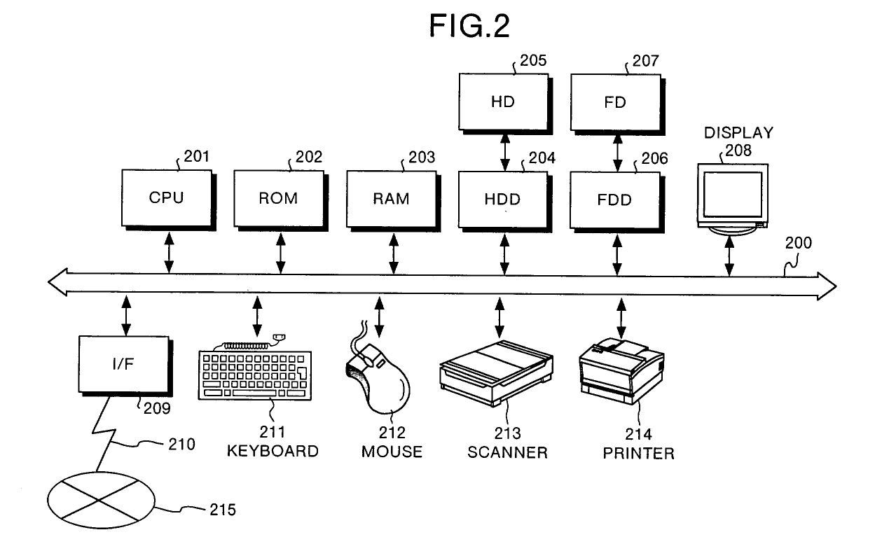

The hardware configuration of the genomic analysis apparatus according to the embodiment of this invention will now be explained. FIG. 2 is a block diagram which shows one example of hardware configuration of the computer 102, being the genomic analysis apparatus according to the embodiment of this invention.

In FIG. 2, the computer 102 comprises a CPU 201, a ROM 202, a RAM 203, an HDD 204, an HD 205, an FDD (flexible disk drive) 206, an FD (flexible disk) 207 as one example of a detachable recording medium, a display 208, an I/F (interface) 209, a keyboard 211, a mouse 212, a scanner 213, and a printer 214. Each component is respectively connected by a bus 200.

The CPU 201 controls the whole computer 102. The ROM 202 stores programs such as a boot program. The RAM 203 is used as a work area of the CPU 201. The HDD 204 controls read/write of data with respect to the HD 205, in accordance with the control of the CPU 201. The HD 205 stores the data written under control of the HDD 204.

The FDD 206 controls read/write of data with respect to the FD 207, in accordance with the control of the CPU 201. The FD 207 stores the data written under control of the FDD 206, or allows the data stored in the FD 207 to be read into an information processing unit. As the detachable recording medium, CD-ROM (CD-R, CD-RW), MO, DVD (Digital Versatile Disk), or memory card may be used, other than the FD 207. The display 208 displays a cursor, an icon or a toolbox, as well as data such as documents, images and functional information. For example, the display may be a CRT, a TFT liquid display, or a plasma display.

The I/F (interface) 209 is connected to a network 100 such as LAN or the Internet through a communication line 210, and connected to other servers and the information processing unit via the network 100. The I/F 209 takes charge of the interface between the network 215 and the inside of the apparatus, and controls input and output of data from and to other servers or information terminal unit. The I/F 209 is for example a modem.

The keyboard 211 has keys for inputting characters, figures and various instructions and inputs data. It may be a touch-panel type input pad or a ten-digit keypad. The mouse 212 performs shift of the cursor or selection of a field, or shift of windows and change of its size. The mouse may be a track ball or a joy stick if it has the similar function as a pointing device.

The scanner 213 optically reads images such as a driver image to take data for the images into the information processing unit. The scanner 213 also has an OCR function, and can read printed genomic sequence information to make it data by the OCR function. The printer 214 prints out image data and document data such as the polymorphic marker information 103. The printer 214 is a laser printer or an ink jet printer.

Functional structure of the genomic analysis apparatus:

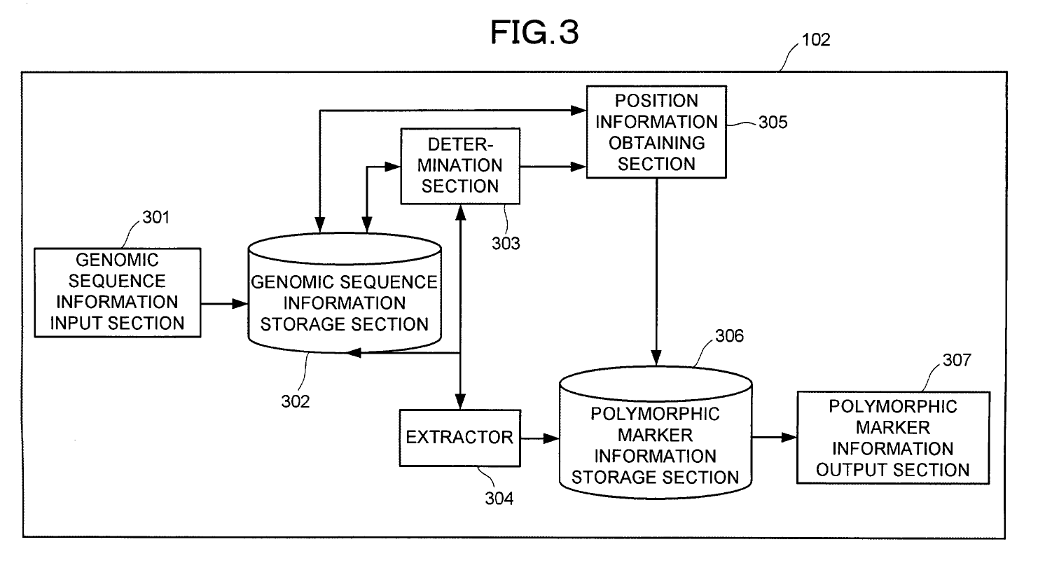

The functional structure of the genomic analysis apparatus will now be explained. FIG. 3 is a block diagram which shows one example of the functional structure of the genomic analysis apparatus according to the embodiment of this invention. In FIG. 3, the genomic analysis apparatus 102 includes a genomic sequence information input section 301, a genomic sequence information storage section 302, a determination section 303, an extractor 304, a position information obtaining section 305, a polymorphic marker information storage section 306 and a polymorphic marker information output section 307.

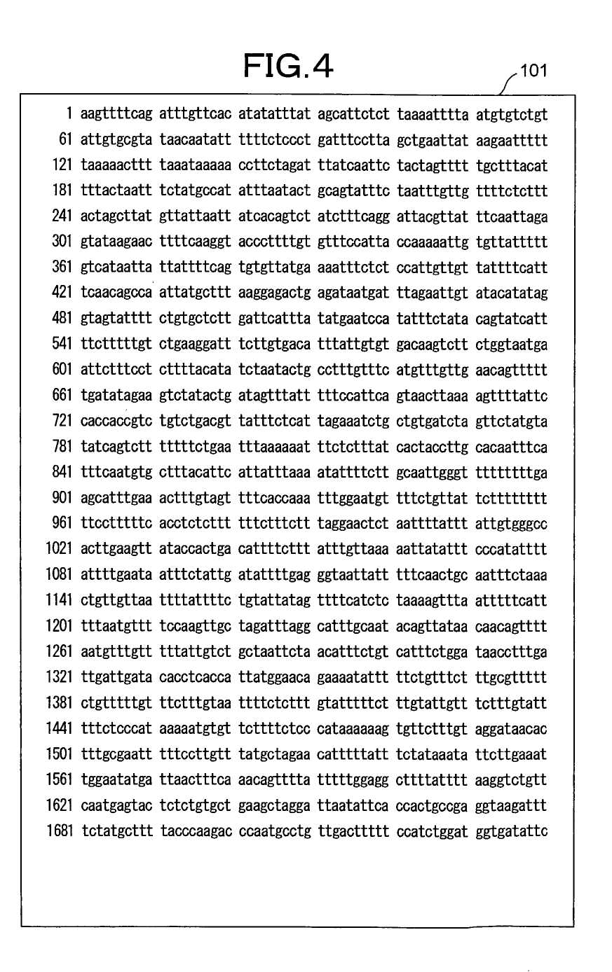

The genomic sequence information input section 301 inputs the genomic sequence information. As shown in FIG. 4 as one example of the information, the genomic sequence information 101 is the information consisting of four bases sequence of adenine (A), thymine (T), guanine (G) and cytosine (C). The genomic sequence information input section 301 realizes its function, for example, by the I/F 209 which receives the genomic sequence information 101 from the network 215. Alternatively, the genomic sequence information input section 301 realizes its function by the FD 207, which is one example of the detachable recording medium that stores the genomic sequence information 101, and the FDD 206. Alternatively, the function may be realized by the scanner 213 having the OCR function, or by the keyboard 211 and the mouse 212.

The genomic sequence information storage section 302 stores the genomic sequence information 101 input through the genomic sequence information input section 301. The genomic sequence information storage section 302 realizes its function by the ROM 202, the RAM 203, the HD 205 and the HDD 204, or the FD 207 and the FDD 206.

The determination section 303 determines whether there is a sequence portion arranged continuously for a plurality of numbers (hereinafter referred to as a “repeat marker”) which is set by either one of the four bases, in the genomic sequence information 101 stored by the genomic sequence storage section 302. For example, it determines whether there is a repeat marker such as “AAAAAAAAAA” or “TTTTTTTTTT” in the genomic sequence information 101. When there is a plurality of repeat markers, all the repeat markers become the object for extracting base sequence information by the extractor 304.

The thus set plurality of numbers is extracted in such a manner that for example 10 or more, that is, one base repeating 10 or more is extracted all from the genomic sequence in terms of accuracy and efficiency. The reason why the number is limited to 10 or more (repetition of 10 or more) is that if the repetition number is small, the polymorphism decreases, and if the repetition number is large, the number of polymorphic markers decreases, and the resolution drops. It is found that repeat markers of 10 or more exist at a frequency of one per about 3000 bases, and it is considered that about one million repeat markers exist in the whole genomic sequence.

When it is determined that there is a sequence portion (repeat marker) in which the same base is arranged continuously for a plurality of numbers, the extractor 304 extracts at least either one of the base sequence information consisting a predetermined number of bases continuously arranged forwards of the repeat marker, and the base sequence information comprising the same number of bases as the predetermined number or a different number of bases arranged continuously rearwards of the repeat marker.

Therefore, the extracted base sequence is the base sequence up to a predetermined number (for example, 300 bases) counted forwards from the base arranged one before of the forefront base of the repeat marker (forward base sequence), and the base sequence information up to a predetermined number (for example, 300 bases) counted rearwards from the base arranged one behind of the last base of the repeat marker (rearward base sequence). The number of the forward base sequence and the number of the rearward base sequence may be the same or different. For example, the number of the forward base sequence may be 400 bases and the number of the rearward base sequence may be 200 bases, or may be the other way round. Further, only the forward base sequence may be extracted, or only the rearward base sequence may be extracted. In either case, the base sequence in the vicinity of the repeat marker has only to be extracted.

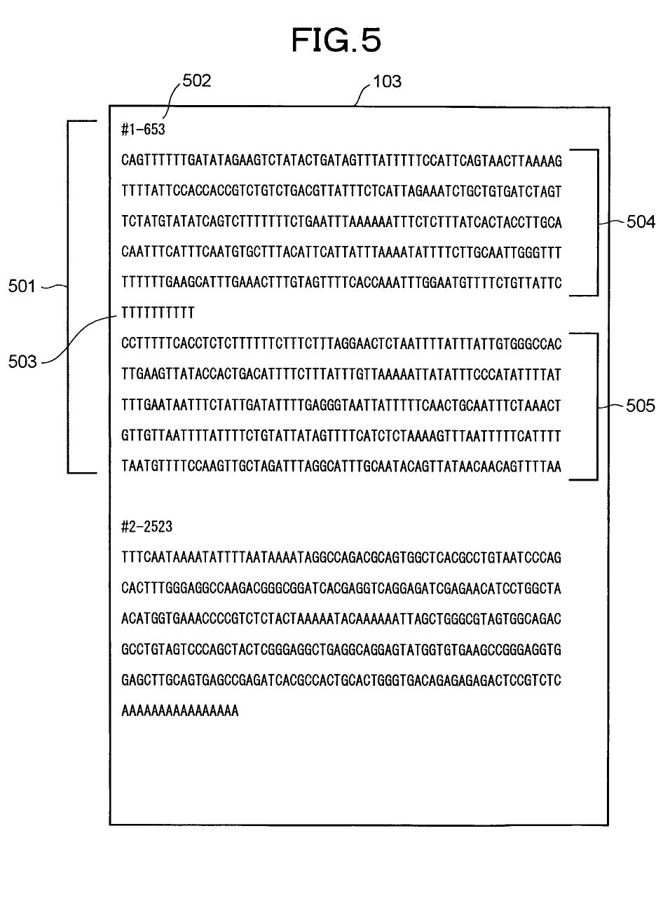

When it is determined that there is a sequence portion (repeat marker) in which a plurality of pieces of the same base is arranged continuously, the position information obtaining section 305 obtains the information related to the position of the repeat marker in the genomic sequence information 101, that is, the information related to which part of the genomic sequence information 101 the repeat marker is positioned in (specifically, information related to a marker name 502 shown in FIG. 5 described below).

FIG. 5 is an explanatory diagram which shows one example of the contents of the polymorphic marker information. In FIG. 5, reference numeral 501 denotes one polymorphic marker information, and 502 is a marker name in the polymorphic marker information 501. “#1-653” which is the marker name 502 indicates that it is the first polymorphic marker and exists in the 653rd base from the head of the genomic sequence information 101, thereby, the position of the polymorphic marker information can be easily identified. Reference numeral 503 denotes a repeat marker, 504 denotes a forward base sequence and 505 denotes a rearward base sequence.

The determination section 303, the extractor 304 and the position information obtaining section 305 realize the functions thereof by the CPU 201 which executes the program stored in the ROM 202, RAM 203, HD 205 or FD 207.

The polymorphic marker information storage section 306 stores the base sequence information extracted by the extractor 304 and the information related to the position obtained by the position information obtaining section 305, as the polymorphic marker information 103. The polymorphic marker information storage section 306 realizes its function by the ROM 202, RAM 203, HD 205 and HDD 204, or FD 207 and FDD 206, as in the genomic sequence information storage section 302.

The polymorphic marker information output section 307 outputs (transmits, displays, or prints) the polymorphic marker information 103 (base sequence information and information related to the position) stored by the polymorphic marker information storage section 306. The polymorphic marker information output section 307 realizes its function by, for example, the FD 207 and FDD 206, the I/F 209, the display 208, or the printer 214 shown in FIG. 2.

Processing procedure of the genomic analysis apparatus:

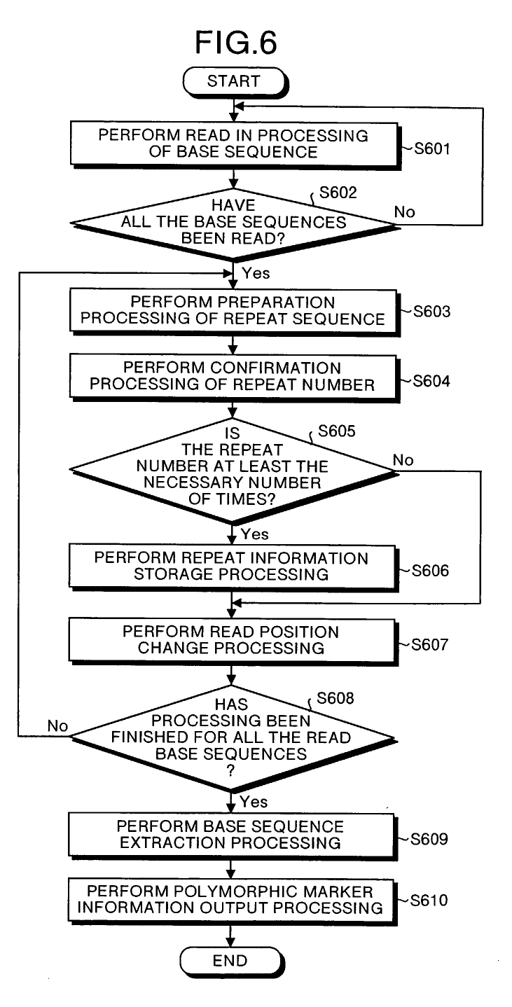

The processing procedure of the genomic analysis apparatus 102 will be explained below. FIG. 6 is a flowchart which shows the processing procedure of the genomic analysis apparatus according to the embodiment of this invention. In the flowchart shown in FIG. 6, at first, the base sequence in the genomic sequence information 101 is read (step S601). Then, it is determined whether all the base sequences have been read (step S602). If all base sequences have not yet been read (step S602: No), control returns to step S601.

Thereafter, when all base sequences have been read (step S602: Yes), repeat sequence is prepared (step S603) to determine the base sequence which becomes a repeat marker. Then, the repeat number of the determined base sequence is confirmed (step S604).

It is determined whether the repeat number of the base sequence is at least a necessary number of times (for example, 10 times), that is, whether the same base continues for the necessary number (step S605), and if the repeat number is not larger than the necessary number of times (step S605: No), control directly proceeds to step S607. On the other hand, if it is at least the necessary number (step S605: Yes), the position of the repeat marker (base sequence) and the information of the repeat number are stored (step S606).

Thereafter, the read-in position of the base sequence is changed (step S607), and the read-in position is advanced further. It is then determined whether the processing has been finished for all the read base sequences (step S608). Here, if it has not finished yet (step S608: No), control returns to step S603, and each step of from step S603 to step S608 is repeated again.

In step S608, when the processing has been finished for all the read base sequences (step S608: Yes), the base sequence information before and after the repeat marker is extracted (step S609). Thereafter, the polymorphic marker information, that is, the repeat marker and the extracted base sequence information of before and after the repeat marker, is output, to perform processing for writing it to the output file 103 (step S610), and the series of processing is finished.

Processing procedure for analysis of disease-related candidate genes:

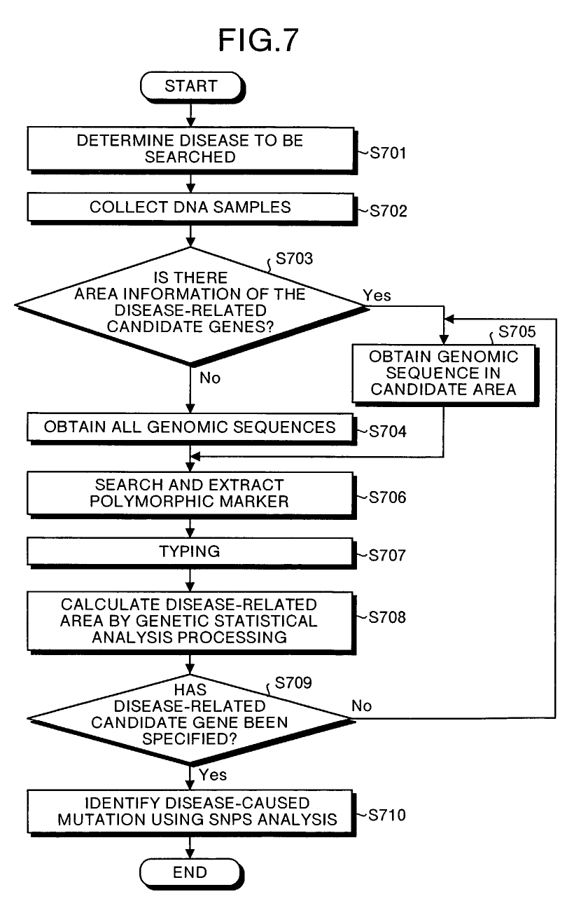

FIG. 7 is a flowchart which shows the processing procedure for analysis of disease-related candidate genes including the genomic analysis method according to the embodiment of this invention. In the flowchart shown in FIG. 7, the disease to be searched is determined first (step S701). The disease to be searched means, for example, diabetes, cancer, or hypertension.

The DNA samples are then collected (step S702). The DNA sample is extracted from blood or the like. At this time, the DNA samples of affected patients and non-affected patients of the objective disease are collected, for example, for 200 patients, respectively. The DNA may be directly gathered from the bloods of all patients, or may be gathered from cells in which peripheral blood B lymphocyte is immortalized (in the state where the lymphocyte can be cultured semi-permanently) by the action of EB virus.

It is then determined whether there is information for the candidate area of the disease-related candidate genes with respect to the objective disease determined in step S703 (step S703). If there is no information for the candidate area of the disease-related candidate genes (step S703: No), the all genomic sequences are obtained (step S704), and control proceeds to step S706. On the other hand, if there is the information for the candidate area of the disease-related candidate genes (step S703: Yes), the genomic sequence in the candidate area is obtained (step S705), and control proceeds to step S706.

In step S706, the polymorphic markers are searched and extracted using the above-described procedure. At this time, at first extraction is roughly performed, and then performed finely in the final stage. Typing is then performed (step S707). That is, the part of each polymorphic marker in each sample is amplified by PCR (polymerase chain reaction), and polymorphism information is experimentally detected by a method such as an SSCP (single strand conformation polymorphism) method or a direct sequence method.

The PCR is a reaction in which a specific sequence of the objective DNA molecule is repetitively reproduced by a certain kind of primer set and heat-resistant DNA polymerase to thereby be amplified. It is an analysis method capable of quantitatively amplifying and detecting a small amount of DNA molecules. The SSCP method is a method of using the fact that single strand DNAs having mutation have different mobility on the gel. The specific contents of typing will be explained later.

Thereafter, the disease-related area is calculated by the processing for genetic statistical analysis (step S708). Specifically, the genetic statistical analysis processing includes, for example, related analysis processing and haplotype analysis processing. All data is analyzed by the computer 107, to search a repeat marker in which the number of repetition agrees with each other as much as possible in the affected patients group, and the number of repetition agrees with each other as much as possible in the non-affected patients group, and the number of repetition does not agree between the affected patients group and the non-affected patients group. It can be determined that there is high possibility that the disease-related candidate gene exists near the marker that satisfies this condition. Known technique can be used for each analysis processing, and hence detailed explanation of each analysis processing is omitted.

It is then determined whether the disease-related candidate gene can be specified (identified) (step S709). If not (step s709: No), control returns to step S705 to obtain the genomic sequence in the candidate area again (step S705), and hereinafter, each step of from step S705 to step S709 is repeated.

On the other hand, in step S709, if the disease-related candidate gene can be specified (step S709: Yes), identification of disease-caused mutation is performed using the SNPs analysis (step S710) to thereby finish the series of processing.

Application example of the polymorphic marker information:

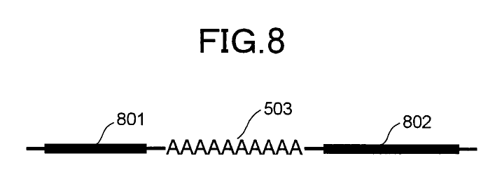

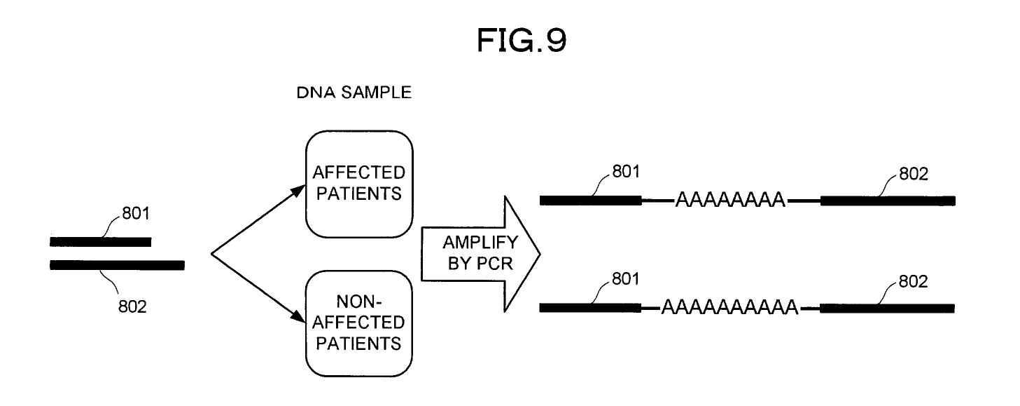

As described above, primer designing is performed from the genomic sequence. The primer designing is to cut out the polymorphic marker, that is, the repeat marker 503 and 300 bases before and after thereof, and determine 20- to 30-base primers (forward primers and reverse primers) within the 300 bases. FIG. 8 and FIG. 9 are explanatory diagrams which show one example of application of the polymorphic marker information. In FIG. 8, reference numeral 801 denotes a forward primer and 802 denotes a reverse primer.

In FIG. 9, when each of the forward primer 801 and the reverse primer 802 of the affected patients and the non-affected patients are amplified by the PCR, a difference occurs in the number of repetition in the repeat marker portion. This difference can be used as a sign, for identifying the disease-related candidate gene.

As described above, according to this embodiment, when the number of SNPs information already found in the target area is small, the disease-related candidate genes can be searched more easily than newly searching the SNPs, in view of time and cost. Further, since the number of polymorphic patterns is small, and the patterns can be used as the same polymorphic marker as the SNPs in the statistical analysis thereafter, it is possible to use the genomic analysis method singly, or add the data of the repeat polymorphic marker to the SNP data to perform simultaneous analysis. That is, the genomic analysis method is very effective as a screening method of genes, for a previous step of the SNPs analysis (pre-SNPs analysis).

In the search of the disease-related candidate genes, when the number of micro-satellite markers is small in the target area, the genomic analysis method according to this embodiment can be used in the same manner as the micro-satellite marker. The micro-satellite is effective in the analysis which uses short generation such as 3 to 5 generations, a so-called family information, but may not be effective in the related analysis using a so-called general group, because there are too many polymorphisms (that is, there are too many mutations). For example, the Japanese group includes a scale of hundreds of thousands of generations, and hence it is difficult to perform grouping, and there are too many contradictions. Therefore, analysis becomes difficult. In view of the combination, the analysis combining the “SNPs” and the analysis according to this embodiment will be better.

Thus, the genome wide is first narrowed by the analysis using the micro-satellite marker, up to about from 3 Gbp to 30 Mbp, wherein bp (base pair) means a base pair. In addition to this method, genome-wide SNPs analysis may also be used. Then, according to the analysis in this embodiment, genes which may be related are picked up to narrow the candidate genes up to about several tens to several. Further, the disease-related candidate gene is identified by the analysis using the SNPs. Since the repeat marker 503 cannot be a direct cause of disease, the analysis is performed finally using the SNPs, to examine which SNPs become the cause.

As the outcome of using this combined analysis, the present inventor has achieved successful results in “Finding of diabetic genes having a significant difference in Japanese”, using the method in this embodiment.

The genomic analysis method in this embodiment may be a computer readable program prepared in advance, and is realized by executing the program on a computer such as a personal computer and a workstation. This program is recorded in a computer readable recording medium such as HD, FD, CD-ROM, MO or DVD, and read out from the recording medium by the computer and executed. This program may be a transmission medium which can be distributed via a network such as the Internet.

As explained above, according to this invention, there is the effect of obtaining the genomic analysis method, the genomic analysis program, the genomic analysis apparatus, and the genomic analysis terminal unit capable of finding a polymorphic marker for identifying a disease-related candidate gene quickly and efficiently with the accuracy close to that of the SNPs, without using the SNPs.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

What is claimed is:

1. A genomic analysis method comprising:
inputting genomic sequence information consisting of four base sequences each having adenine (A), thymine (T), guanine (G) and cytosine (C);
determining whether there is a sequence portion in which any one of the four bases is arranged continuously for a plurality of numbers, in the input information;
extracting, when it is determined that there is the sequence portion, at least one of base sequence information consisting of a predetermined number of bases continuously arranged forwards of the sequence portion and base sequence information consisting of the same number of bases as the predetermined number or a different number of bases which are continuously arranged rearwards of the sequence portion; and
outputting the extracted base sequence information.

2. The genomic analysis method according to claim 1, further comprising obtaining the information related to a position of the sequence portion in the genomic sequence information when it is determined there is the sequence portion, wherein
the outputting step includes outputting the obtained information related to the position.

3. The genomic analysis method according to claim 1, wherein the determination step includes determining whether there is a sequence portion in which any one of the four bases is arranged continuously for at least 10, in the input genomic sequence information.

4. A genomic analysis program which allows a computer to execute, the genomic analysis program comprising:
inputting genomic sequence information consisting of four base sequences each having adenine (A), thymine (T), guanine (G) and cytosine (C);
determining whether there is a sequence portion in which any one of the four bases is arranged continuously for a plurality of numbers, in the input information;
extracting, when it is determined that there is the sequence portion, at least one of base sequence information consisting of a predetermined number of bases continuously arranged forwards of the sequence portion and base sequence information consisting of the same number of bases as the predetermined number or a different number of bases which are continuously arranged rearwards of the sequence portion; and
outputting the extracted base sequence information.

5. The genomic analysis program according to claim 4, which allows the computer to execute, the genomic analysis program further comprising obtaining the information related to the position of the sequence portion in the genomic sequence information when it is determined that there is the sequence portion, wherein
the outputting step includes outputting the obtained information related to the position.

6. The genomic analysis program according to claim 4, wherein the determination step includes determining whether there is a sequence portion in which any one of the four bases is arranged continuously for at least 10, in the input genomic sequence information.

7. A genomic analysis apparatus comprising:
an input unit which inputs genomic sequence information consisting of four base sequences each having adenine (A), thymine (T), guanine (G) and cytosine (C);
a determination unit which determines whether there is a sequence portion in which any one of the four bases is arranged continuously for a plurality of numbers, in the input information;
an extraction unit which extracts, when it is determined that there is the sequence portion, at least one of base sequence information consisting of a predetermined number of bases continuously arranged forwards of the sequence portion and base sequence information consisting of the same number of bases as the predetermined number or a different number of bases which are continuously arranged rearwards of the sequence portion; and
an output unit which outputs the extracted base sequence information.

8. The genomic analysis apparatus according to claim 7, further comprising an obtaining unit which obtains the information related to a position of the sequence portion in the genomic sequence information when it is determined that there is the sequence portion, wherein
the output unit outputs the obtained information related to the position.

9. The genomic analysis apparatus according to claim 7, wherein the determination unit determines whether there is a sequence portion in which any one of the four bases is arranged continuously for at least 10, in the input genomic sequence information.
